(19) United States
(12) Patent Application Publication
(10) Pub. No.: US 2024/0353383 A1
(43) Pub. Date: Oct. 24, 2024

(54) MULTILAYER SENSOR FOR SELECTIVELY DETECTING A VOC VAPOR

(71) Applicant: FreshAir Sensor, LLC, Lebanon, NH (US)

(72) Inventors: Matthew CURTIN, Lebanon, NH (US); Lauren BROCK, Lebanon, NH (US)

(21) Appl. No.: 18/636,151

(22) Filed: Apr. 15, 2024

Related U.S. Application Data
(60) Provisional application No. 63/460,530, filed on Apr. 19, 2023.

(51) Int. Cl. G01N 33/00; G01N 27/12

(52) U.S. Cl. CPC G01N 33/0047; G01N 27/126; G01N 33/0031

(57) ABSTRACT

The present disclosure relates to a sensor for selectively detecting a volatile organic compound (VOC) vapor. The sensor comprises an insulating substrate; a pair of interdigitated electrodes disposed on the insulating substrate; a sensing layer of a semi-conductive material disposed on the pair of interdigitated electrodes, the sensing layer exhibiting an electrical property that is dependent on the concentration of the VOC vapor to which the sensing layer is exposed; and a filtering layer of a polymer or metal organic framework disposed on the sensing layer, the filtering layer enhancing the selectivity of the sensing layer to the VOC vapor. The present disclosure also relates to a system for selectively sensing a VOC vapor comprising a plurality of these sensors, a method for selectively detecting a VOC vapor by using these sensors, and a method for producing such sensors.

### CROSS-REFERENCE TO RELATED APPLICATIONS

This application claims benefit of priority to U.S. Provisional Application No. 63/460,530, filed Apr. 19, 2023; which is herein incorporated by reference in its entirety.

### BACKGROUND

Semiconducting metal oxide-based gas sensors have been used for detecting the presence of volatile organic compounds (VOC) in the environment. However, these metal oxide-based gas sensors are responsive to many different gases and lack the selectivity to differentiate between different gases they are exposed to, especially between the more hazardous contaminants and safer chemicals. Formaldehyde is one specific example of a particularly hazardous chemical that is used in a variety of manufacturing processes.

Another challenge to the conventional semiconducting metal oxide-based gas sensors, is that these sensors often operate at elevated temperatures of up to 400° C. Such high operational temperatures of these sensor systems increase the complexity and power consumption of these sensor systems. High operational temperatures can also negatively impact the lifetime of the sensors.

At present, no sensors are capable of selectively detecting specific VOC vapor, such as an aldehyde, in an environment where other VOC vapors, such as a non-aldehyde VOC vapor, are present.

### SUMMARY

The present disclosure provides devices, systems, and methods for the selectively detecting a volatile organic compound (VOC) vapor, such as an aldehyde vapor.

In one aspect, the disclosure provides a sensor for selectively detecting a volatile organic compound (VOC) vapor. The sensor comprises an insulating substrate; a pair of interdigitated electrodes disposed on the insulating substrate; a sensing layer of a semi-conductive material disposed on the pair of interdigitated electrodes, the sensing layer exhibiting an electrical property that is dependent on the concentration of the VOC vapor to which the sensing layer is exposed; and a filtering layer of a polymer or metal organic framework disposed on the sensing layer, the filtering layer enhancing the selectivity of the sensing layer to the VOC vapor.

In some embodiments, the electrical property exhibited by the sensing layer is conductivity, capacitance, or inductance. In some embodiments, the electrical property of the sensing layer increases or decreases by different levels with increasing concentration of different VOC vapors to which the sensing layer is exposed. In some embodiments, the electrical property of the sensing layer increases or decreases by a first level with increasing concentration of an aldehyde and the electrical property of the sensing layer increases or decreases by a second level with increasing concentration of a non-aldehyde VOC vapor, the first level being different from the second level.

In some embodiments, the non-aldehyde VOC vapor is water, an alcohol, acetone, or ammonia. In some embodiments, the aldehyde is formaldehyde, acetaldehyde, or glutaraldehyde.

In some embodiments, the semi-conductive material of the sensing layer is a metal salt or metal oxide. In one embodiment, the semi-conductive material of the sensing layer is a metal chloride. In one embodiment, the semi-conductive material of the sensing layer is tin oxide (SnO₂). In some embodiments, the metal salt or metal oxide is doped with a metal different than the metal in the metal salt or the metal oxide.

In some embodiments, the filtering layer enhances the selectivity of the sensing layer to the VOC vapor by modifying the surface chemistry of the sensing layer, thereby modifying the interaction of the VOC vapor with the sensing layer. In some embodiments, the filtering layer comprises a molecularly imprinted polymer. In some embodiments, the filtering layer comprises a non-conductive metal organic framework. In some embodiments, the filtering layer comprises ZIF-7, ZIF-8, ZIF-67, ZIF-L, HKUST-1, and/or UIO-66.

In some embodiments, the insulating substrate comprises glass or glass fiber, resin, silicon, rubber, sapphire, nitride, carbide ceramic, a printed circuit board, or a combination thereof.

In some embodiments, the interdigitated electrodes comprise nickel, chromium, gold, copper, palladium, platinum, lead, silver, tin, or a combination thereof. In some embodiments, the surface of the interdigitated electrode is pretreated with an acid, base, or surfactant to increase adhesion to the insulating substrate and/or the sensing layer.

In some embodiments, the sensor further comprises a second filtering layer of a polymer or metal organic framework disposed in between the pair of interdigitated electrodes and the sensing layer, the filtering layer enhancing the selectivity of the sensing layer to the VOC vapor.

In some embodiments, the semi-conductive material of the sensing layer is tin oxide (SnO₂); the filtering layer comprises a non-conductive metal organic framework; and the VOC vapor is an aldehyde and the electrical property of the sensing layer increases or decreases with increasing concentration of the aldehyde to which the sensing layer is exposed; wherein the degree of the electrical property increase or decrease of the sensor, when being exposed to the aldehyde, is higher than the degree of the electrical property increase or decrease of the same sensor, when being exposed to a non-aldehyde VOC vapor.

In some embodiments, the sensor is operated at a temperature of 30° C. or lower.

In another aspect, the disclosure provides a system for selectively sensing a volatile organic compound (VOC) vapor, comprising a plurality of sensors. Each sensor comprises an insulating substrate; a pair of interdigitated electrodes disposed on the insulating substrate; a sensing layer of a semi-conductive material disposed on the pair of interdigitated electrodes, the sensing layer exhibiting an electrical property that is dependent on the concentration of the VOC vapor to which the sensing layer is exposed; and a filtering layer of a polymer or metal organic framework disposed on the sensing layer, the filtering layer enhancing the selectivity of the sensing layer to the VOC vapor.

In some embodiments, each sensor further comprises a second filtering layer of a polymer or metal organic framework disposed in between the pair of interdigitated electrodes and the sensing layer, the filtering layer enhancing the selectivity of the sensing layer to the VOC vapor.

In some embodiments, the system further comprises a control unit, wherein for each sensor of the plurality of sensors, the control unit induces an electric current to flow through each sensor; and generates a concentration measurement of the VOC vapor based on the electrical property.

In some embodiments, the electrical property exhibited by the sensing layer is conductivity, capacitance, or inductance. In some embodiments, the electrical property of the sensing layer increases or decreases by different levels with increasing concentration of different VOC vapors to which the sensing layer is exposed. In some embodiments, the electrical property of the sensing layer increases or decreases by a first level with increasing concentration of an aldehyde and the electrical property of the sensing layer increases or decreases by a second level with increasing concentration of a non-aldehyde VOC vapor, the first level being different from the second level.

In some embodiments, the non-aldehyde VOC vapor is water, an alcohol, acetone, or ammonia. In some embodiments, the aldehyde is formaldehyde, acetaldehyde, or glutaraldehyde.

In yet another aspect, the disclosure provides a method for selectively detecting a volatile organic compound (VOC) vapor. The method comprises exposing a sensor to an environment in need of detecting the VOC vapor, the sensor comprising an insulating substrate, a pair of interdigitated electrodes disposed on the insulating substrate, a sensing layer of a semi-conductive material disposed on the pair of interdigitated electrodes, the sensing layer exhibiting an electrical property that is dependent on the concentration of the VOC vapor to which the sensing layer is exposed, and a filtering layer of a polymer or metal organic framework disposed on the sensing layer, the filtering layer enhancing the selectivity of the sensing layer to the VOC vapor. The sensor has a VOC vapor detection limit that spans a detection range. The method also comprises generating a signal when the sensor is exposed to a sample of the VOC vapor having a gas density, gas concentration, or gas partial pressure greater than or equal to the VOC vapor detection threshold of the sensor.

In some embodiments, the sensor further comprises a second filtering layer of a polymer or metal organic framework disposed in between the pair of interdigitated electrodes and the sensing layer, the second filtering layer enhancing the selectivity of the sensing layer to the VOC vapor.

In some embodiments, the VOC vapor is an aldehyde. In one embodiment, the VOC vapor is formaldehyde, acetaldehyde, or glutaraldehyde.

In some embodiments, the VOC vapor detection threshold is selected from the group consisting of 10 ppb, 100 ppb, 1 ppm, 10 ppm, 25 ppm, 50 ppm, 100 ppm, and 250 ppm of the VOC compound.

In some embodiments, the method is carried out at a temperature of 30° C. or lower.

In yet another aspect, the disclosure provides a method for producing a sensor for selectively detecting a volatile organic compound (VOC) vapor. The method comprises depositing a pair of interdigitated electrodes on an insulated substrate; depositing a sensing solution to form a sensing layer on the pair of electrodes, wherein the sensing solution comprises a semi-conductive nanoparticle dissolved or suspended in an organic solvent; and depositing a filtering solution on the sensing layer to form a filtering layer on the sensing layer, wherein the filtering solution comprises a polymer or metal organic framework dissolved or suspended in an organic solvent.

In some embodiments, the method further comprises, prior to the depositing the sensing solution, depositing a filtering solution on the sensing layer to form a filtering layer on the pair of electrodes, wherein the filtering solution comprises a polymer or metal organic framework dissolved or suspended in an organic solvent.

In some embodiments, the organic solvent for the sensing solution or the filtering solution is ethanol, chloroform, acetone, dichloromethane, or dimethylformamide. In some embodiments, wherein the organic solvent is ethanol.

In some embodiments, the interdigitated electrodes comprise nickel, chromium, gold, copper, palladium, platinum, or a combination thereof. In one embodiment, the interdigitated electrodes comprise chromium.

In some embodiments, the step of depositing the sensing solution or depositing the filtering solution is carried out by spin-coating, dip-coating, ink-jet printing, plotting, or a combination thereof.

Alternatively, in each of the above aspects, the sensor may have a different layout of the layers. For instance, the sensor for selectively detecting a VOC vapor may comprises: an insulating substrate;
a pair of interdigitated electrodes disposed on the insulating substrate;
a filtering layer of a polymer or metal organic framework disposed on the pair of interdigitated electrodes; and
a sensing layer of a semi-conductive material disposed on the filtering layer, the sensing layer exhibiting an electrical property that is dependent on the concentration of the VOC vapor to which the sensing layer is exposed,
wherein the filtering layer enhancing the selectivity of a sensing layer to the VOC vapor.

### BRIEF DESCRIPTION OF THE DRAWINGS

The features and advantages of the present disclosure will become apparent from a consideration of the following detailed description presented in connection with the accompanying drawings in which:

FIG. 1A shows a cross-sectional view of an exemplary selective VOC sensor according to one embodiment of the disclosure. FIG. 1B shows a cross-sectional view of an exemplary selective VOC sensor according to an alternative embodiment of the disclosure. FIG. 1C shows a cross-sectional view of an exemplary selective VOC sensor according to an alternative embodiment of the disclosure.

FIG. 2 shows a schematic of an exemplary system for selectively sensing a VOC according to one embodiment of the disclosure.

FIG. 3 is a flow chart illustrating a method for selectively detecting a VOC vapor according to one embodiment of the disclosure.

FIG. 4 is a flow chart illustrating a method for forming an exemplary selective VOC sensor according to one embodiment of the disclosure.

### DETAILED DESCRIPTION

The disclosure provides an economic way to selectively monitor the presence of a specific VOC vapor (e.g., an aldehyde) in the environment.

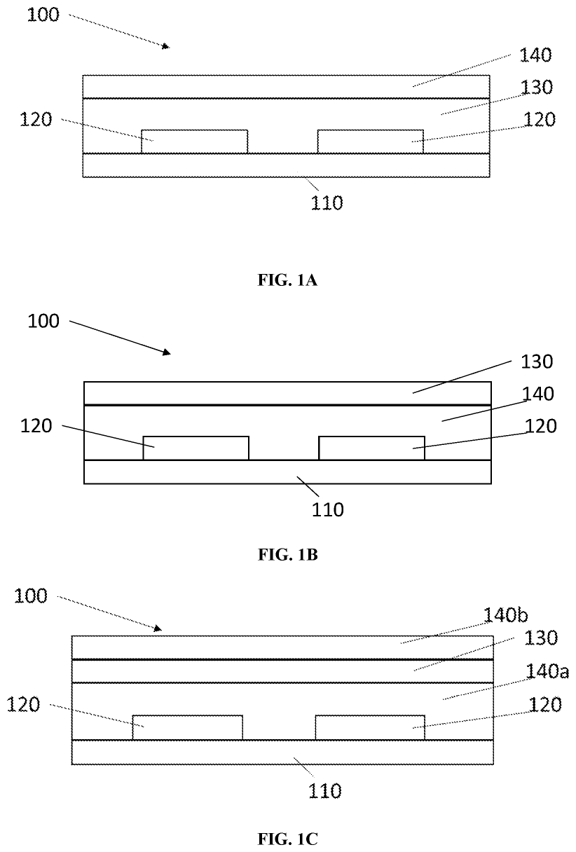

FIG. 1A shows an exemplary sensor 100 for selectively detecting a VOC vapor comprising an insulating substrate 110, a pair of interdigitated electrodes 120 disposed on the insulating substrate, a sensing layer of a semi-conductive material 130 disposed on the pair of the interdigitated electrodes, and a filtering layer of a polymer or metal organic framework 140 disposed on the sensing layer according to an embodiment.

FIG. 1B shows an exemplary sensor 100 for selectively detecting a VOC vapor comprising an insulating substrate 110, a pair of interdigitated electrodes 120 disposed on the insulating substrate, a filtering layer of a polymer or metal organic framework 140 disposed on the pair of interdigitated electrodes, and a sensing layer of a semi-conductive material 130 disposed on the filtering layer according to an alternative embodiment.

FIG. 1C shows an exemplary sensor 100 for selectively detecting a VOC vapor comprising an insulating substrate 110, a pair of interdigitated electrodes 120 disposed on the insulating substrate, a filtering layer of a polymer or metal organic framework 140a disposed on the pair of interdigitated electrodes, a sensing layer of a semi-conductive material 130 disposed on the filtering layer according to an alternative embodiment, and another filtering layer of a polymer or metal organic framework 140b disposed on the sensing layer.

In these figures, the insulating substrate 110 may be prepared from glass or glass fiber; a resin such as epoxy resin, polyimide resin, phenol resin, novolak resin, or the like; silicon; rubber; sapphire; nitride; carbide ceramic; a printed circuit board, or a combination thereof. In some embodiments, the insulating substrate is a glass substrate or a substrate made of a composite material comprising glass or glass fiber. In one embodiment, the insulating substrate is a composite of epoxy resin reinforced with glass fiber, e.g., a FR-4 epoxy glass laminate. In some embodiments, the insulating substrate is a polyimide resin such as Kapton. The interdigitated electrodes 120 may comprise, for example, nickel, chromium, gold, copper, palladium, platinum, lead, silver, tin, or a combination thereof. In one example of fabrication, the interdigitated electrodes 120 are formed of chromium metal and is deposited on a glass substrate or a substrate made of a composite material comprising glass or glass fiber 110. The surface of the interdigitated electrode 120 may be pretreated before use. In some embodiments, the surface of the interdigitated electrode is pretreated with an acid, base, or surfactant to increase adhesion to the insulating substrate and/or the sensing layers.

The sensing layer 130 comprises a semi-conductive material. In some embodiments, the semi-conductive material of the sensing layer 130 is a metal salt or metal oxide. The metal(s) in the metal salts may be an alkali metal, an alkaline earth metal, a transition metal, lanthanide metal, or combinations thereof. The anionic species for the metal salts may be salts of a hydrochloric acid, carboxylic acid, phosphoric acid, phosphorous acid, hydrogen phosphoric acid, or boric acid. In some embodiments, the metal salt or metal oxide is doped with a metal different than the metal in the metal salt or the metal oxide. In one embodiment, the semi-conductive material of the sensing layer 130 is tin oxide (SnO₂). In one embodiment, the semi-conductive material of the sensing layer 130 is a metal chloride.

The sensing layer 130 may have a thickness ranging between 5 nm to 50 microns, for instance, between 10 nm to 50 microns, between 100 nm to 50 microns, between 500 nm to 50 microns, between 1 microns to 50 microns, between 5 microns to 50 microns, between 1 micron to 40 microns, between 1 micron to 30 microns, between 1 micron to 30 microns, between 1 micron to 20 microns, between 1 micron to 10 microns, or between 5 micron to 10 microns. In one embodiment, the sensing layer 130 has a thickness ranging between 5 micron to 10 microns.

The sensing layer 130 exhibits an electrical property that is dependent on the concentration of the VOC vapor to which the sensing layer is exposed. The electrical property may be conductivity, capacitance, or inductance. The electrical property of the sensing layer 130 may increase or decrease by different levels with increasing concentration of different VOC vapors to which the sensing layer is exposed. In some embodiments, provided herein is a sensor for selectively detecting an aldehyde. The sensing layer has the ability to differentiate between an aldehyde and a non-aldehyde VOC vapor. Any aldehyde known in the art can be detected by the sensor 100 described herein. Exemplary aldehydes are formaldehyde, methylformcel, butylformcel, acetaldehyde, propionaldehde, butyraldehyde, crotonaldehyde, valeraldehyde, caproaldehyde, heptaldehyde, glutaraldehyde, and benzaldehyde. In some embodiments, the aldehyde is formaldehyde, acetaldehyde, or glutaraldehyde. “Non-aldehyde VOC” refers to any VOC compound that is not an aldehyde. In some embodiments, the non-aldehyde VOC compound to be distinguished from the aldehyde by the sensor is water, an alcohol, acetone, or ammonia.

The filtering layer 140 enhances the selectivity of the sensing layer 130 to the VOC vapor it is exposed to. For instance, the filtering layer 140 can enhance the selectivity of the sensing layer 130 to a VOC vapor by modifying the surface chemistry of the sensing layer 130, thereby modifying the interaction of the VOC vapor with the sensing layer. In one embodiment, the filtering layer 140 can enhance the selectivity of the sensing layer 130 to an aldehyde by modifying the surface chemistry of the sensing layer 130, thereby modifying the interaction of the aldehyde with the sensing layer. In some embodiments, the filtering layer comprises a molecularly imprinted polymer. In some embodiments, the filtering layer comprises a non-conductive metal organic framework. In some embodiments, the filtering layer comprises ZIF-7, ZIF-8, ZIF-67, ZIF-L, HKUST-1, and/or UIO-66.

As shown in FIGS. 1A-1C, one or more filtering layers 140 can be used in the sensor, and the filtering layer(s) can have different configuration relative to the sensing layer 130. For instance, as shown in FIG. 1A, the filtering layer 140 can be deposited on the sensing layer 130; alternatively, as shown in FIG. 1B, the filtering layer 140 can be deposited below the sensing layer 130, in between the pair of interdigitated electrodes and the sensing layer. When a filtering layer is deposited below the sensing layer, it can perform a strengthening role to increase the durability of the sensing film.

In some embodiments, one or more additionally filtering layer can be included herein to form the sensor. In some embodiments, a second filtering layer of a polymer or metal organic framework may be disposed in between the pair of interdigitated electrodes and the sensing layer, wherein the second filtering layer enhances the selectivity of the sensing layer to the VOC vapor. For instance, as shown in FIG. 1C, the sensor has one filtering layer 140a deposited below the sensing layer 130, in between the pair of interdigitated electrodes and the sensing layer, and one filtering layer 140b deposited on the sensing layer 130. Additional filtering layers can be added on top of 140b, which can be controlled to adjust the sensitivity of the sensor to different detection limits.

When multiple filtering layers are used in the sensor, each of the filtering layers may be the same or different materials. For instance, in one embodiment, one filtering layer can comprise a molecularly imprinted polymer and another filtering layer can comprise a metal organic framework. In one embodiment, different filtering layers may contain different polymers. In one embodiment, different filtering layers may contain different metal organic framework materials.

In some embodiments, the electrical property of the sensing layer 130 increases or decreases by a first level with increasing concentration of an aldehyde, and the electrical property of the sensing layer increases or decreases by a second level with increasing concentration of a non-aldehyde VOC vapor, wherein the first level is different from the second level. In some embodiments, the increase or decrease of the first level (associated with increasing concentration of an aldehyde) is larger than the increase or decrease of the second level (associated with increasing concentration of a non-aldehyde VOC vapor). The difference in the level of increase or decrease can distinguish the detection of the aldehyde over a non-aldehyde VOC vapor. In some embodiments, the increase or decrease of the first level (associated with increasing concentration of an aldehyde) is at least 5%, 10%, 15%, 20%, 25%, 30%, 35%, 40%, 45%, 50%, 55%, 60%, 65%, 70%, 75%, 80%, 85%, 90%, 95%, or 100% larger than the increase or decrease of the second level (associated with increasing concentration of a non-aldehyde VOC vapor). In some embodiments, the increase or decrease of the first level (associated with increasing concentration of an aldehyde) is at least 1.5-fold, 2-fold, 2.5-fold, 3-fold, 3.5-fold, 4-fold, 4.5-fold, 5-fold, 6-fold, 7-fold, 8-fold, 9-fold, or 10-fold larger than the increase or decrease of the second level (associated with increasing concentration of a non-aldehyde VOC vapor).

In some embodiments, the electrical property of the sensing layer 130 increases by a first level with increasing concentration of an aldehyde, whereas the electrical property of the sensing layer 130 decreases by a second level with increasing concentration of a non-aldehyde VOC vapor. In some embodiments, the electrical property of the sensing layer 130 decreases by a first level with increasing concentration of an aldehyde, whereas the electrical property of the sensing layer 130 increases by a second level with increasing concentration of a non-aldehyde VOC vapor.

In some embodiments, provided herein is a sensor for selectively detecting an aldehyde, that is, the sensor has the ability to differentiate between an aldehyde and a non-aldehyde VOC vapor. In these aldehyde sensors, the semi-conductive material of the sensing layer is tin oxide (SnO₂); the filtering layer comprises a non-conductive metal organic framework; and the VOC vapor is an aldehyde and the electrical property of the sensing layer increases or decreases with increasing concentration of the aldehyde to which the sensing layer is exposed. These aldehyde sensor shows a much higher sensitivity to an aldehyde than a non-aldehyde VOC compounds. Without being bound by theory, the configuration and materials of the sensing layer and filtering layer together bring in enhanced sensitivity/selectivity to the detection of the aldehyde. Although some other sensors involving using titanium dioxide may have shown response to VOC compounds, they are not selective to aldehydes. For instance, the degree of the electrical property increase or decrease of the sensor, when being exposed to the aldehyde, is higher than the degree of the electrical property increase or decrease of the same sensor, when being exposed to a non-aldehyde VOC vapor.

In some embodiments, provided herein is a sensor for selectively detecting an aldehyde, that is, the sensor has the ability to differentiate between an aldehyde and a non-aldehyde VOC vapor. In some embodiments, the degree of the differentiation of the aldehyde sensor between an aldehyde and a non-aldehyde VOC vapor, when being operated at a temperature of 50° C. or lower, at 40° C. or lower, at 30° C. or lower, at 25° C. or lower, or at 20° C. or lower, is higher than the degree of the degree of the differentiation of the same aldehyde sensor, when being operated at a temperature of 100° C. or higher, at 200° C. or higher, at 300° C. or higher, or at 400° C. or higher. Thus, the aldehyde sensor is more advantageous to a commercially available sensor (e.g., those metal oxide gas sensor such as the titanium dioxide gas sensor) that requires a heater to function, as the aldehyde sensor disclosed herein not only can be operated at room temperature or near room temperature (e.g., at 20-30° C.) and does not require heating, but provides a better sensitivity at room temperature or near room temperature.

FIG. 2 shows a system 200 for selectively sensing a VOC vapor that includes a plurality of sensors 210, each being an example of the sensor 100 of FIGS. 1A-IC. The plurality of sensors includes N sensors, labeled 100 (1), 100 (2), . . . 100 (N), and each sensor includes an insulating substrate; a pair of interdigitated electrodes disposed on the insulating substrate; a sensing layer of a semi-conductive material disposed on the pair of interdigitated electrodes, the sensing layer exhibiting an electrical property that is dependent on the concentration of the VOC vapor to which the sensing layer is exposed; and a filtering layer of a polymer or metal organic framework disposed on the sensing layer, the filtering layer enhancing the selectivity of the sensing layer to the VOC vapor. Each of the sensors 100 (1), 100 (2), . . . , and 100 (N), is a respective example of the sensor 100 of FIGS. 1A-1C. Thus, all the above descriptions and embodiments relating to the sensor 100 and its elements apply to FIG. 2. In some embodiments, the electrical property of the sensor dependent on the concentration of the VOC vapor is conductivity, capacitance, or inductance. In some embodiments, the electrical property of the sensing layer of each sensor increases or decreases by different levels with increasing concentration of different VOC vapors to which the sensing layer is exposed. In some embodiments, each of the sensors 100 (1), 100 (2), . . . , and 100 (N) is a sensor for selectively detecting an aldehyde. That is, the sensor system can be used to differentiate between an aldehyde and a non-aldehyde VOC vapor. In some embodiments, the aldehyde is formaldehyde, acetaldehyde, or glutaraldehyde. In some embodiments, the non-aldehyde VOC vapor is water, an alcohol, acetone, or ammonia. In some embodiments, the electrical property of the sensing layer in each of the sensors 100 (1), 100 (2), . . . , and 100 (N) increases or decreases by a first level with increasing concentration of an aldehyde and the electrical property of the sensing layer in each of the sensors 100 (1), 100 (2), . . . , and 100 (N) increases or decreases by a second level with increasing concentration of a non-aldehyde VOC vapor, the first level being different from the second level.

In some embodiments, the sensing system 200 further comprises a control unit 240 that, for each of the sensors 100 (1), 100 (2), . . . , and 100 (N), induces an electrical current 242 to flow through each of the sensors 100 (1), 100 (2), . . . , and 100 (N) and generates a concentration measurement 244 of the VOC vapor based at least in part on the electrical property of the sensing layer in each of the sensors 100 (1), 100 (2), . . . , and 100 (N). In one embodiment, the concentration measurements 244 may be stored in an onboard storage 250 communicatively coupled to the control unit 240. In one embodiment, each of the sensors 100 (1), 100 (2), . . . , and 100 (N) is configured to have a different detection threshold. For instance, the sensing system 200 may include a plurality of sensors with a respective plurality of detection thresholds, such that each detection threshold corresponds to a different concentration of the VOC vapor (e.g., an aldehyde) (e.g., ≥10 ppb, ≥100 ppb, ≥1 ppm, ≥10 ppm, ≥25 ppm, ≥50 ppm, ≥100 ppm, and ≥250 ppm).

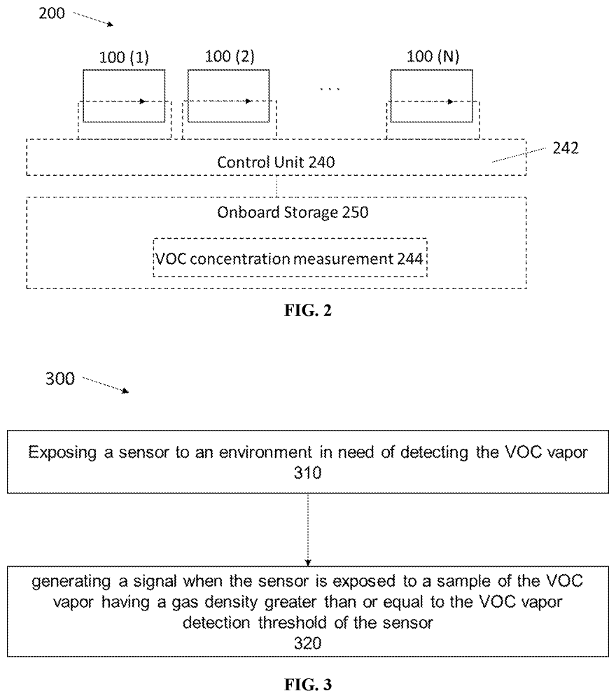

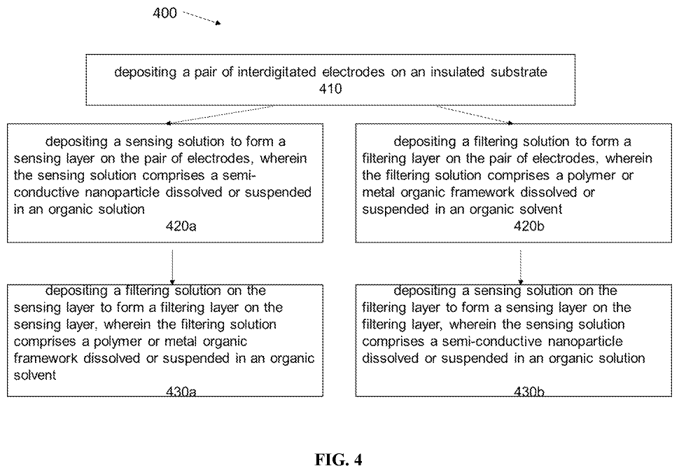

FIG. 3 is a flow chart illustrating a method 300 for selectively detecting a volatile organic compound vapor. Method 300 may be used in conjunction with the sensor 100, and/or the sensing system 200, as disclosed in FIGS. 1A-1C and FIG. 2. In step 310, a sensor (e.g., 100 as described in FIGS. 1A-1C), or a sensing system comprising a plurality of sensors (e.g., 200 and 100 (1) . . . 100 (N) as described in FIG. 2), comprising an insulating substrate, a pair of interdigitated electrodes disposed on the insulating substrate, a sensing layer of a semi-conductive material disposed on the pair of interdigitated electrodes, the sensing layer exhibiting an electrical property that is dependent on the concentration of the VOC vapor to which the sensing layer is exposed, and a filtering layer of a polymer or metal organic framework disposed on the sensing layer, the filtering layer enhancing the selectivity of the sensing layer to the VOC vapor is provided. The sensor (or the plurality of the sensors) is exposed to an environment in need of detecting a VOC vapor. All the above descriptions and embodiments relating to the sensor 100 and sensing system 200 and their respective elements apply to the method described herein, e.g., in FIG. 3. In step 320, a signal is generated if the sensor (or the plurality of the sensors) is exposed to a sample of the VOC vapor having a gas density, gas concentration, or gas partial pressure greater than or equal to the VOC vapor detection threshold of the sensor (or the plurality of the sensors). The sensor (or the plurality of the sensors) has a VOC vapor detection limit that span a detection range. In some embodiments, the VOC vapor is an aldehyde, and the method 300 is for selectively detecting the aldehyde. In some embodiments, the VOC vapor is formaldehyde, acetaldehyde, or glutaraldehyde, and the method 300 is for selectively detecting formaldehyde, acetaldehyde, or glutaraldehyde. In some embodiments, the VOC vapor detection limit may be 10 ppb, 100 ppb, 1 ppm, 10 ppm, 25 ppm, 50 ppm, 100 ppm, or 250 ppm of that specific VOC compound (e.g., formaldehyde, acetaldehyde, or glutaraldehyde).

In some embodiments, the VOC vapor is an aldehyde, and the method 300 is for selectively detecting the aldehyde, and the method is carried out at a temperature of 50° C. or lower, at 40° C. or lower, at 30° C. or lower, at 25° C. or lower, or at 20° C. or lower. In some embodiments, the method is carried out at about 30° C. In some embodiments, the sensors are operated at room temperature. In some embodiments, the method is carried out at room temperature.

FIG. 4 is a flow chart illustrating a method 400 for producing a selective VOC sensor. Method 400 may be used to form the sensor 100 as described in FIGS. 1A-1C and/or the sensing system 200 as described in FIG. 2. Step 410 involves depositing a pair of interdigitated electrodes on an insulated substrate. Step 420a involves depositing a sensing solution on the pair of electrodes to form a sensing layer on the pair of electrodes, wherein the sensing solution comprises a semi-conductive nanoparticle dissolved or suspended in a solvent, such as water or an organic solvent. Step 430a involves depositing a filtering solution on the sensing layer to form a filtering layer on the sensing layer, wherein the filtering solution comprises a polymer or metal organic framework dissolved or suspended in an organic solvent. Step 420b involves depositing a filtering solution on the pair of electrodes to form a filtering layer on the pair of electrodes, wherein the filtering solution comprises a polymer or metal organic framework dissolved or suspended in a solvent, such as water or an organic solvent. Step 430b involves depositing a sensing solution on the filtering layer to form a sensing layer on the filtering layer, wherein the sensing solution comprises a semi-conductive nanoparticle dissolved or suspended in a solvent, such as water or an organic solvent. Additional steps of depositing a filtering solution can be applied for multiple times to deposit multiple layers of filtering layers, as discussed above, to form the configuration, for instance, as shown in FIG. 1C. All the above descriptions and embodiments relating to the sensor 100 and sensing system 200 and their respective elements apply to the method described herein, e.g., in FIG. 4. In some embodiments, the interdigitated electrodes comprise nickel, chromium, gold, copper, palladium, platinum, lead, silver, tin, or a combination thereof. In one example of fabrication, the interdigitated electrodes are formed of chromium metal and is deposited on a glass substrate or a substrate made of a composite material comprising glass or glass fiber. The organic solvent for the sensing solution or the filtering solution may include methanol, ethanol, chloroform, acetone, acetonitrile, n-methyl-2-pyrrolidone, dichloromethane, or dimethylformamide. In one embodiment, the organic solvent for the sensing solution or the filtering solution is ethanol. In some embodiments, the filtering solution comprises a non-conductive metal organic framework (e.g., ZIF-7, ZIF-8, ZIF-67, ZIF-L, HKUST-1, and/or UIO-66) dissolved or suspended in an organic solvent. The sensing solution or filtering solution may be deposited onto the surface of the electrodes by spin-coating, dip-coating, ink-jet printing, plotting, or a combination thereof. In one embodiment, the depositing step is carried out by spin-coating.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

### Examples

The following examples are for illustrative purposes of the present disclosure only and are not intended to limit the present invention in any way. Equivalents or substitutes are within the scope of the present invention.

### Example 1: Preparation of Imprinted Polymer Films by Evaporation

A 0.145 g/mL tin oxide solution was made by adding tin (IV) oxide nanopowder to a vial, adding ethanol in 5 parts, and sonicating the mixtures for one minute in between additions. The ink was then stirred overnight before use. A separate 0.075 g/mL ZIF-8 solution in ethanol was made following the same procedure as the procedure for the preparation of the tin oxide solution.

Interdigitated electrodes (chromium metal on glass substrate) were cleaned using a microfiber cloth to remove particulates. Then, 50 μL of the tin oxide solution, prepared as described above, was deposited onto the surface of the electrode using a spin coater at 1500 rpm for 30 seconds. After drying, 200 μL of the ZIF-8 solution, prepared as described above, was deposited onto the surface of the electrodes that were previously coated with the tin oxide, using a spin coater at 1500 rpm for 30 seconds.

1. A sensor for selectively detecting a volatile organic compound (VOC) vapor, comprising:
an insulating substrate;
a pair of interdigitated electrodes disposed on the insulating substrate;
a sensing layer of a semi-conductive material disposed on the pair of interdigitated electrodes, the sensing layer exhibiting an electrical property that is dependent on the concentration of the VOC vapor to which the sensing layer is exposed; and
a filtering layer of a polymer or metal organic framework disposed on the sensing layer, the filtering layer enhancing the selectivity of the sensing layer to the VOC vapor.

2. The sensor of claim 1, wherein the electrical property is conductivity, capacitance, or inductance.

3. The sensor of claim 1, wherein the electrical property of the sensing layer increases or decreases by a first level with increasing concentration of an aldehyde and the electrical property of the sensing layer increases or decreases by a second level with increasing concentration of a non-aldehyde VOC vapor, the first level being different from the second level.

4. The sensor of claim 3, wherein the non-aldehyde VOC vapor is water, an alcohol, acetone, or ammonia.

5. The sensor of claim 3, wherein the aldehyde is formaldehyde, acetaldehyde, or glutaraldehyde.

6. The sensor of claim 1, wherein the semi-conductive material of the sensing layer is a metal salt or metal oxide.

7. The sensor of claim 6, wherein the semi-conductive material of the sensing layer is tin oxide (SnO₂).

8. The sensor of claim 1, wherein the filtering layer enhances the selectivity of the sensing layer to the VOC vapor by modifying the surface chemistry of the sensing layer, thereby modifying the interaction of the VOC vapor with the sensing layer.

9. The sensor of claim 1, wherein the filtering layer comprises a non-conductive metal organic framework.

10. The sensor of claim 9, wherein the filtering layer comprises ZIF-7, ZIF-8, ZIF-67, ZIF-L, HKUST-1, and/or UIO-66.

11. The sensor of claim 1, further comprising a second filtering layer of a polymer or metal organic framework disposed in between the pair of interdigitated electrodes and the sensing layer, the second filtering layer enhancing the selectivity of the sensing layer to the VOC vapor.

12. The sensor of claim 1, wherein:
the semi-conductive material of the sensing layer is tin oxide (SnO₂);
the filtering layer comprises a non-conductive metal organic framework; and
the VOC vapor is an aldehyde and the electrical property of the sensing layer increases or decreases with increasing concentration of the aldehyde to which the sensing layer is exposed;
wherein the degree of the electrical property increase or decrease of the sensor, when being exposed to the aldehyde, is higher than the degree of the electrical property increase or decrease of the same sensor, when being exposed to a non-aldehyde VOC vapor.

13. A system for selectively sensing a volatile organic compound (VOC) vapor, comprising:
a plurality of sensors, each sensor according to claim 1.

14. The system of claim 13, further comprising a control unit, wherein for each sensor of the plurality of sensors, the control unit:
induces an electric current to flow through each sensor; and
generates a concentration measurement of the VOC vapor based on the electrical property.

15. A method for selectively detecting a volatile organic compound (VOC) vapor, comprising:
exposing a sensor to an environment in need of detecting the VOC vapor, the sensor comprising
an insulating substrate,
a pair of interdigitated electrodes disposed on the insulating substrate,
a sensing layer of a semi-conductive material disposed on the pair of interdigitated electrodes, the sensing layer exhibiting an electrical property that is dependent on the concentration of the VOC vapor to which the sensing layer is exposed, and
a filtering layer of a polymer or metal organic framework disposed on the sensing layer, the filtering layer enhancing the selectivity of the sensing layer to the VOC vapor; and
generating a signal when the sensor is exposed to a sample of the VOC vapor having a gas density, gas concentration, or gas partial pressure greater than or equal to the VOC vapor detection threshold of the sensor;
wherein the sensor has a VOC vapor detection limit that spans a detection range.

16. The method of claim 15, wherein the method is carried out at a temperature of 30° C. or lower.

17. A method for producing a sensor for selectively detecting a volatile organic compound (VOC) vapor, comprising:
depositing a pair of interdigitated electrodes on an insulated substrate;
depositing a sensing solution to form a sensing layer on the pair of electrodes, wherein the sensing solution comprises a semi-conductive nanoparticle dissolved or suspended in an organic solvent; and
depositing a filtering solution on the sensing layer to form a filtering layer on the sensing layer, wherein the filtering solution comprises a polymer or metal organic framework dissolved or suspended in an organic solvent.

18. The method of claim 17, further comprising, prior to the depositing the sensing solution, depositing a filtering solution on the sensing layer to form a filtering layer on the pair of electrodes, wherein the filtering solution comprises a polymer or metal organic framework dissolved or suspended in an organic solvent.

19. The method of claim 17, wherein the organic solvent for the sensing solution or the filtering solution is ethanol, chloroform, acetone, dichloromethane, or dimethylformamide.

20. The method of claim 17, wherein the step of depositing the sensing solution or depositing the filtering solution is carried out by spin-coating, dip-coating, ink-jet printing, plotting, or a combination thereof.
